(19) United States
(12) Patent Application Publication
(10) Pub. No.: US 2019/0336572 A1
(43) Pub. Date: Nov. 7, 2019

(54) COMPOSITION AND METHOD FOR ORAL DELIVERY OF STABLE FORMULATIONS OF COBRA VENOM

(71) Applicant: Paul F. Reid, Sunrise, FL (US)

(72) Inventor: Paul F. Reid, Sunrise, FL (US)

(21) Appl. No.: 15/972,188

(22) Filed: May 6, 2018

(51) Int. Cl. A61K 38/17; C07K 14/46; A61P 29/00; A61K 9/00

(52) U.S. Cl. CPC A61K 38/17; A61K 9/0053; A61P 29/00; C07K 14/46

(57) ABSTRACT

A composition of sterile cobra venom and a method for its oral administration to provide significant analgesic effects to a human and/or animal are disclosed. Such cobra venom compositions comprise a sterilized solution preserved by the addition of one or more suitable food-grade preservatives. The venom composition may be conveniently administered orally by means of a metered spray device.

### FIELD OF THE INVENTION

This invention relates generally to the field of pharmaceutical and healthcare products for the treatment of pain, and more particularly to stable formulations of sterile cobra venom suitable for oral administration, and products comprising these formulations in liquid and spray forms.

### BACKGROUND OF THE INVENTION

Millions of people around the world suffer from untreated pain related to a variety of illnesses and ailments, as well as from unidentified causes. Humans have searched for effective painkillers for many, many years. Natural pain-killing compositions have been discovered from various sources as varied as willow bark, opium poppies (a source of morphine, codeine, and thebaine), and snake venoms. Opium, for example, was used as a narcotic by Hippocrates, introduced to Persia and India by Alexander the Great, and used as a painkiller by Paracelsus during the Renaissance.

Despite their effectiveness as analgesics, opiate drugs such as morphine and codeine are classified as narcotics and their use is subject to complex legal and medical regulations in most countries. Furthermore, opiate drugs have a high potential for addiction and abuse.

Clinical investigations from the 1930's through the 1950's revealed that cobra venom is a potent pain killer with activity superior to morphine, but without the known adverse effects of opiates. In the United States, injectable cobra venom for medicinal use was available up to the late 1970s. In homeopathy, the preferred dilution of cobra venom is a 1:10,000 dilution, although the volume actually administered has been quite variable over the years. In 1870's Europe, the preferred dose was 10⁻⁴ (0.1 mg/mL). Present guidelines, as provided in the Homeopathic Pharmacopoeia of the United States (USHP), list the recommended dilutions in the range of 10⁻⁶ to 10⁻⁸.

In Chinese medicine, the venom is prepared on demand and small quantities, usually sufficient for one week, are given to the patient. Alternatively, the dried venom is mixed into lactose (triturated) and provided as small pills. The venom solution is then mixed with tea or water. The dosage to be used is left to the discretion of the treating physician.

Unfortunately, at these low dilutions, the direct ingestion of cobra venom left subjects with unpleasant side effects that included irritated and sore throat, headache, nausea, vomiting, abdominal cramps and pain, sudden bowel movements and diarrhea. Given the existence of such problematic side effects, not surprisingly the utilization of oral cobra venom as a pain remedy declined and was ultimately abandoned in Western medicine.

The use of cobra venom as a treatment for pain enjoyed a short resurgence in the 1930's following research and clinical studies that revealed that cobra venom had very potent analgesic activity. However, during this period cobra venom was administered only by injection, requiring that the venom solution be rendered sterile prior to use. This was accomplished by prolonged exposure of the venom to heat in the range of 60° Celsius. The instability of these cobra venom solutions for injection influenced the Council on Pharmacy and Chemistry as reported in Journal of the American Medical Association of 1940 on the inclusion of cobra venom solution as a New and Non-official Remedy. The report referenced ten ampoules of cobra venom solution in a small box were submitted but they showed broad differences in color and, on standing, that different amounts of precipitate was seen. The manufacturer's attention was called to this lack of uniformity, and the Council was informed that the differences were due to the preservative used. Van Esveld reported in 1935 that a preserved saline solution of processed cobra venom was stable for at least 9 months at room temperature particularly if the solution was acidified and stored in the dark. However, the injection of acidic solutions can be quite uncomfortable.

Considering the favorable outcomes related to pain relief reported for injectable cobra venom in trials conducted during the 1930's to the 1970's, although associated with side-effects and possible stability issues, what are needed are compositions and methods for providing analgesic levels of cobra venom in a stable form suitable for self-administration by a patient in need of the substantial pain relief that cobra venom may provide and free of the side-effects which have previously limited its use.

### SUMMARY OF THE INVENTION

The present invention relates to a novel formulation of cobra venom, and methods for the administration of cobra venom. More particularly, the invention provides formulations of sterile solutions of cobra venom containing a stabilizing agent that are suitable for oral administration in several forms, including as beverages and oral sprays that can be used with an oral delivery device to permit convenient, metered administration of the venom. The resulting solutions and delivery systems are safe for the storage and administration of cobra venom over extended periods of time.

In one aspect, the invention provides a composition of cobra venom suitable for oral administration. Such a composition comprises a sterile cobra venom solution admixed with a food-grade preservative. In various aspects, the preservative may be chosen from among the group consisting of sodium benzoate, potassium sorbate, and combinations thereof.

In various aspects of the invention, a sterile cobra venom solution may be formulated at a final concentration of 0.01 to 1 mg/mL. Such concentrations generally may contain from about 0.1 to about 0.5 mg/ml, and from about 0.01 to about 0.05 mg/ml, respectively.

In another aspect, the invention provides a method for the oral administration of a composition comprising a sterile cobra venom solution and a food-grade preservative, the method comprising administering the composition as a spray or jet. Some aspects of the invention provide a healthcare product comprising a solution of sterile cobra venom admixed with a food-grade preservative, the venom in the solution having a formulation of from about 1 mg/ml to about 0.01 mg/mL, and a metered pump configured to deliver a volume of the solution in the range of from about 0.05 to about 1 ml.

In various aspects of the invention, a composition is provided as a beverage. Compositions may also be provided for release from edible films, for example, which may be placed on the mucosa within the mouth.

### DETAILED DESCRIPTION OF THE INVENTION

The present invention relates to a stable formulation of cobra venom, and methods for the oral administration of cobra venom. More particularly, the invention provides formulations of sterile solutions of cobra venom admixed with one or more stabilizing polyol(s), the combination of venom and polyol(s) being suitable for injectable, topical and oral administration in several forms, including, for example, as beverages, oral sprays, lozenges, and edible films that can be used with an oral delivery device to permit convenient, metered administration of the venom. The resulting solutions and delivery systems provide safe storage and administration of cobra venom over extended periods of time.

Formulations of the product as described and claimed herein have been determined to be effective for the reduction of chronic pain symptoms, such as chronic back pain, in human subjects. These analgesic benefits are delivered without significant adverse effects and without potential for addiction, representing a significant advance over opioid-based analgesics.

Cobra venom preparations may be made from the venom of the Asian cobra (e.g., Naja tripudians) and related species according to methods provided in the United States and European Homeopathic Pharmacopoeias. Historically, cobra venoms were selected by homeopathic physicians based upon their neurotoxic activity for treatment of disorders of the nervous system. Without intending to be bound by theory, it is therefore believed that the principal active components in the venoms were most likely neurotoxins. In contrast to the opioid drugs which bind to opioid receptors (G-protein coupled receptors acting by GABAnergic neurotransmission), the venom neurotoxins are known to primarily target the cholinergic system by blocking the activity of acetylcholine, although it is possible that other receptors or targets may be involved in the analgesic effect. More recently, it has been determined that orally active component of cobra venom is Cardiotoxin and that, based on the low toxicity by the oral and topical route, the neurotoxins are principally active when injected.

Preparing an injectable form of a cobra venom solution is now straightforward. However, developing a convenient and effective stable venom formulation that avoided the known problems associated with usual routes of administration presented several challenges. It is thought that the commonly used metacresol preservative may have contributed to the stability issues of injectable cobra venom formulation in the 1940s. Toxicology studies in mice were conducted by the inventor, from which it was determined that mice could drink a 1 mg/ml solution of cobra venom for 28 days with a daily intake of 350 mg/kg. By contrast, injection of merely 10-12 micrograms was a fatal dose. The mice in this study gained weight, were quite active, and were apparently unaffected by the ingestion of cobra venom at this concentration via their drinking water. This perplexing and unexpected finding led the inventor to question why the side effects of oral administration of cobra venom as described in the prior art from the last century, being so to detrimental in humans, were seemingly absent in his studies with the mice. This data also suggested that an oral and injectable form would exert their analgesic effects through differing mechanisms.

Providing a sterile composition for oral administration is facilitated by the addition of a food-grade preservative to the venom (i.e., at least one food-grade preservative). The inventor has determined that the use of such a preservative has no adverse effect on the efficacy of the venom composition for achieving analgesia after oral administration. Suitable food-grade preservatives include, for example, sodium benzoate, and potassium sorbate, with the use of other suitable preservatives being known to those of skill in the art and within the scope of the present invention, given the present disclosure.

Protein instability is one of the major reasons why protein pharmaceuticals are administered traditionally through injection rather than taken orally like most small chemical drugs. Protein pharmaceuticals usually have to be stored under cold conditions or freeze-dried to achieve an acceptable shelf life (Wang W. Instability, stabilization, and formulation of liquid protein pharmaceuticals. Int J Pharm. 1999 Aug. 20; 185(2):129-88). Oxidation is one of the major chemical degradation pathways for protein pharmaceuticals. Methionine, cysteine, histidine, tryptophan, and tyrosine are the amino acid residues most susceptible to oxidation due to their high reactivity with various reactive oxygen species (Li S, Schoneich C, Borchardt R T. Chemical instability of protein pharmaceuticals: Mechanisms of oxidation and strategies for stabilization. Biotechnol Bioeng, 1995 Dec. 5; 48(5):490-500). Oxidation during protein processing and storage can be induced by contaminating oxidants, catalyzed by the presence of transition metal ions and induced by light. Oxidative modification depends on the structural features of the proteins as well as the particular oxidation mechanisms inherent in various oxidative species, and may also be influenced by pH, temperature, and buffer composition. Oxidation of a critical residue at or near the enzyme active site or receptor binding site, or a dramatic change in the structure of the protein upon oxidation may be the molecular basis for the altered bioactivity in the oxidation products of protein.

Strategies to stabilize proteins against oxidation can be classified into intrinsic methods (site-directed mutagenesis and chemical modification), physical methods (solid vs. liquid formulations) and use of chemical additives (Li S, Schoneich C, Borchardt R T. Chemical instability of protein pharmaceuticals: Mechanisms of oxidation and strategies for stabilization. Biotechnol Bioeng, 1995 Dec. 5; 48(5):490-500). Predicting a priori the alteration of pharmaceutical properties caused by the three degradation routes is difficult, and must be determined on a case-by-case basis for each protein. The difficulty in predicting the effect of degradation and analyzing the temperature dependence of reaction rates in proteins results in longer development times for protein formulations than for small molecule formulations Cleland J L, Powell M F, Shire S J. The development of stable protein formulations: a close look at protein aggregation, deamidation, and oxidation. Crit Rev Ther Drug Carrier Syst 1993; 10(4):307-77).

The use of polyols in liquid oral and parenteral cobra venom formulations had not been described. Topical formulations have described formulations with polyols (i.e. menthol) though not necessarily for the purposed of stabilizing the active pharmaceutical ingredient. The most common of polyol stabilizers are glycerol and propylene glycol. Propylene glycol is particularly useful as it is commonly used as an antimicrobial preservative; it may be used as a preservative in parenteral formulations but, due to its very bitter taste, not as an oral preservative.

The preparation of injectable and oral formulas of cobra venom for the simplest composition possible required careful consideration and activity testing was necessitated by the unpredictable activities of the polyol stabilizers in addition to the issues of excipient interaction. Ethanol as a parenteral preservative may stabilize some drug actives but it will precipitate proteins in cobra venom. Glycerol may be useful as a stabilizer in other injectable and oral formulas but its potential negative impacts on kidney function precludes its chronic use. For injectable solutions of cobra venom the stabilizing and preservative effects off propylene glycol were chosen. It has been determined that a cobra venom solution in saline with 30% propylene glycol can be stored refrigerated in excess of 10 years without loss of potency or precipitation. Additionally, topical formulas of cobra venom that utilize propylene glycol with or without glycerol are also very stable, having a room temperature shelf life in excess of 5 years as determined clinically.

Oral formulations for widespread public use have similar limitations. It is best to avoid lactose and sucrose in subjects with lactose intolerance and diabetes respectively. As xylitol demonstrated stabilizing effects on cobra venom it was chosen as the sweetening agent but given its propensity to induce gastrointestinal distress its strength must be keep to a minimum. It was determined through laboratory and clinical testing that a cobra venom formula for oral administration using xylitol at 5% with sodium benzoate as a preservative retains its potency for over 5 years at room temperature while still providing a palatable solution.

Without intending to be bound by theory, it may be that the clinical tests on humans described in the homeopathic literature, which were conducted with orally administered cobra venom, were flawed because the venom product may have been contaminated with bacteria. Furthermore, the dosing of the material employed a concentration that was too high. Today, it is possible to remove such bacterial contamination by sterile filtration without compromising the quality of the venom. When cobra venom is prepared under sterile conditions, no abdominal problems are experienced. Sterile filtration may be accomplished, for example, using a filter having a pore size of 0.45 μm or smaller. Sterilization may also be accomplished by prolonged low-heat treatment or by pasteurization. Heating has been found to enhance the analgesic and anti-inflammatory active of the venom though the considerable precipitation of non-active proteins makes large-scale filtration difficult for the production of an oral or topical formula. Heat processing would be preferred for injectable formulas in order to reduce the levels of higher molecular weight proteins thereby providing a less immunologically reactive solution. It has been discovered that the pre-administration of antihistamines can prevent allergic reactions or permit the administration of the cobra venom solution to a subject already sensitized to the drug.

Throat irritation had previously been consistently reported when cobra venom was taken by mouth. In the 1873 copy of Guy's Hospital Report, Taylor reported that he received 4.2 grains (273 mg) of dried cobra venom and, allowing for the loss of water through desiccation, it represented half a drachm (1.78 g) or 15% of the venom. Clinically, cobra venom was used at strengths of 1 mg/mL. The volumes or quantities administered then were quite small, often consisting of a pill or a drop of the diluted tincture. However, side effects associated with this form of administration caused the characteristic irritated and sore throat associated with cobra venom administration.

In conventional Western medicine, a defined quantity of a drug is administered to a patient in need thereof. For example, 1 mg of a drug can be given in a volume of 1 ml or 10 ml. In homeopathic medicine, however, the same volume of material is administered, regardless of the dilution factor. Furthermore, the dilution factors that used are quite large, ten-fold at a minimum, and more commonly 100-fold or 1000-fold in order to reach the exceedingly high dilutions that are routinely used by homeopathic doctors.

With the goal of achieving a standard oral dose of the active ingredient, the inventor produced a therapeutically effective dose (e.g., 1 mg in 1 ml, if starting with a tincture having a concentration of 10 mg/ml) that could be administered in dosages such as a single dose in a 10-fold greater volume, or as 10×0.1 mL doses, or as 100×0.01 mg/mL doses. Use of these dilutions might reduce the possibility of experiencing the adverse effects that caused this medication to be abandoned as a potent orally-administered analgesic. Tests were therefore conducted as described in the Examples below, using several liquid formulations based from 10 mg/mL to 0.02 mg/mL. Results of clinical testing with human subjects with back pain showed effective reduction of back pain using dilutions of cobra venom stock solutions corresponding to each of these homeopathic doses. Positive results were achieved with a minimum of side effects reported previously using other dosage ranges.

From the standpoint of modern drug manufacturing, formulations in the 10 mg/mL to 0.010 mg/mL range were shown to provide several advantages over prior art formulations. For example, preparing the venom as a diluted liquid solution facilitates the preparation of a sterile product, and easier handling by automated systems. The handling of dried cobra venom poses a risk of aerosolization leading to intoxication and hypersensitivity.

The addition of a suitable edible preservative permits the sterile solution to be dispensed into containers for long-term storage and prevents the solution from becoming adulterated during the period of use. A metered spray or jet permits the venom to be administered orally as a controlled dose that allows frequent administration with limited esophageal irritation. The formulation and metered dose permits the venom solution to be administered over periods of days to weeks. These formulations may be useful for pain relief in both humans and/or animals.

The inventor has also discovered that a certain degree of effectiveness appears to accompany administering the venom to the mucous membranes of the mouth, such as would be achieved by an oral spray. This may also be accomplished, along with the additional benefit of potentially providing modified release compositions, using edible films such as those known to those of skill in the art. Such films and dissolvable strips have been made, for example, using pullulan, whey proteins, and other carbohydrates and proteins known to those of skill in the art of pharmaceutical formulation and administration. Reformulation of the injectable and oral solutions would be required and appropriately tested to confirm stability as suggested by Cleland et al., 1993 due to the inconsistency associated with stabilization by polyols.

The invention may be further described by means of the following non-limiting examples.

### EXAMPLES

### Example 1

In order to determine the optimal cobra venom formulations for parenteral administration, stability assessments were undertaken. Under accelerated storage conditions, it was confirmed that unpreserved and unstabilized solutions of cobra venom maintain their stability equally in saline or water over 75 days. Three batches of the parenteral drug formulations each at 0.1 mg/ml in 0.9% saline and preserved with 0.007% BZK were prepared. Accelerated studies with injectable formulas employing benzalkonium chloride as a preservative yielded a protein precipitate. A cobra venom solution was prepared by heating venom to 60° C. for 3 hours. It has been determined that a cobra venom solution in saline with 30% propylene glycol can be stored refrigerated in excess of 10 years without loss of potency or precipitation.

### Example 2. Topical Formulation of Cobra Venom

The formulation of the gel consists mainly of penetration enhancers that also represent established preservatives. A retrospective investigation of gel formulations revealed that the formulation was stable and, when stored in air tight containers, the constituents did not separate, discolor or decompose. An examination of two early commercial batches of cobra venom produced in March of 2010 and stored at 19-25° C. showed no signs of deterioration nor separation when examined in July of 2012 (28 months). Additionally topical formulas of cobra venom that utilize propylene glycol with or without glycerol are also very stable, having a room temperature shelf life in excess of 5 years as determined clinically.

### Example 3. Oral Formulation of Cobra Venom

Oral formulations of cobra venom at 0.035 mg/ml preserved with methyl paraben (0.2%) were established and studied under accelerated conditions (49° C. for 3 months) representing a reaction rate 12 fold greater than that of storage at 25° C. The oral formulations included citric acid, sodium citrate, xylitol and flavoring. Formulations of cobra venom with each product excipient and in certain combinations was prepared in duplicate and one sample stored at ambient (19-25° C.) conditions and a second at elevated temperatures (48-51° C.). Within 19 days it was determined that sodium citrate by itself adversely affected the potency of cobra venom and needed to be removed from the formulation. Sodium citrate was later found to destabilize Crotoxin, a neurotoxin from Crotalus durissus venom. Additionally, cobra venom oral formulas with methyl paraben were unstable. It was also determined that the polyol xylitol appeared to enhance stability at a strength of 5%, a lower level than predicted by computer modelling.

Formulations of cobra venom in water, with strengths of 0.035 mg/mL, 0.14 mg/mL and 0.28 mg/mL, in addition to 5% xylitol, 1% flavoring, 0.2% sodium benzoate, 0.1% potassium hydrogen phosphate with a pH ranging from 4.0-7.0 were prepared and tested for stability at 49 degrees Celsius. These formulations were found to be stable and to surprisingly retain their biological activity for at least 3 months, equivalent to 3 years as determined from the Arrhenius equation.

Formulations of cobra venom in water, with strengths of 0.035 mg/mL, 0.14 mg/mL and 0.28 mg/mL, in addition to 5% xylitol, 1% flavoring, 0.2% sodium benzoate, 0.1% potassium hydrogen phosphate with a pH ranging from 4.0-7.0 were prepared and tested for stability at 25 and 30 degrees Celsius. These formulations were found to be stable and to surprisingly retain their biological activity for at least 5 years.

### Example 4. Potency Testing of Cobra Venom Formulas

The procedure serves to efficiently indicate the potency or toxicity of specific venom agents. The time to death is recorded in addition to the symptoms displayed by the animal. This helps quantify the level of the toxicity retained in the sample being examined. For cobra venom strengths of less than 0.1 mg/ml inject 0.5 mL and observe the animals for 24 hrs. Two out of three animals must succumb to the venom in order to pass. With strengths in excess of 0.1 mg/ml, inject 0.5 ml and observe for 3 hours. Two out of three animals must succumb for the assay to pass.

The mouse is preferred—any strain and genotype of rodent is acceptable including wild type, outbred species. Age must be in excess of 6 weeks and less than 6 months. The sex of the animal is not critical to the assay and mixed populations are allowed. Usually three animals are used for potency/toxicity. Inspect the animal for suitability for inclusion in study. Animals with obvious difficulties such a low activity, ruffled coat and injuries should be excluded.

Fill syringe with required volume of sample and remove any residual air. Remove animal from cage and inject preferably intraperitoneally or subcutaneously the test substance in a slow but deliberate pace, thus minimizing discomfort to the animal. Mark the animal if necessary with ear punch or colored pens and replace in the cage. Repeat with all animals as necessary while observing previously injected animals.

Observe mice closely for 20 minutes and then every hour thereafter up to a minimum of 8 hours. If animal dies during this period, note time of death. Check on the animals following overnight exposure and continue observation of the animals for a total of 24 hours.

### Example 5. Activity of Native Cobra Venoms in Animal Models of Pain

A study set out to establish if analgesia or anti-inflammatory inflammation could be exerted by the parenteral and oral administration of cobra venom and identify if there are significant interspecies differences in the activities of the venoms and characterize the likely active agents. Employing the commonly used mouse models of pain: formalin, hot-plate and acetic acid writhing tests, this study sought to compare the safety and efficacy of orally and parenterally administered cobra venom. The pharmacodynamic activity of venoms from several species of cobra was examined. In the acetic acid writhing assay, the activity of the venoms was compared to the opiate standard, morphine. The results revealed that the intraperitoneal and intragastric administration of venoms from either N. siamensis, N. naja, N. atra, N. kaouthia, and O. hannah, had analgesic effects in mice with an excellent therapeutic window. Attempts to estimate the circulating levels of neurotoxins utilizing a receptor binding assay failed. However, interpretation of the toxicity data in addition to the highly variable levels of alpha neurotoxins between cobra venom species suggested that the active component were not neurotoxins but another venom component. Surprisingly, oral cobra venoms at 100 mcg/Kg suppressed writhing 6 hours into the study comparably to that of oral morphine at 5000 mcg/Kg at 1 hour into the study.

### Example 6. Activity of Pasteurized Venom in Animal Models of Pain

Employing the commonly used mouse models of pain: formalin, hot-plate and acetic acid writhing tests, a study sought to compare the safety and efficacy of orally and parenterally administered cobra venom that was pasteurized. The pharmacodynamic activity of venoms from several species of cobra was examined. In the acetic acid writhing assay, the activity of the venoms was compared to the opiate standard, morphine. The results revealed that, contrary to published reports, the oral administration of pasteurized N. kaouthia venom (Nyloxin) had anti-inflammatory and analgesic effects. Oral pasteurized venom at 100 mcg/Kg suppressed writhing 6 hours into the study comparably to that of oral morphine at 5000 mcg/Kg at 1 hour into the study.

### Example 7. Parenteral Administration of Stabilized Cobra Venom

In 2015, a female subject with chronic hip pain that was not responsive to oral administration of cobra venom was administered pasteurized cobra venom stabilized with 30% propylene glycol by injection. The administered drug was 9 years old. It was administered subcutaneously for 6 months. Pain relief was noted after 3 weeks of treatment. When therapy ceased at 6 months, the hip pain did not recur. During the treatment period the subject developed an anaphylactic reaction to the cobra venom. It was found that the pre-administration of antihistamines (diphenhydramine) could prevent allergic reactions and permitted treatment to continue.

### Example 8. Topical Administration of Cobra Venom

A batch of cobra venom gel (0.06 mg/mL) was provided to a former NFL player with severely arthritic hands. The topical formula, comprised of 20% glycerol and 20% propylene glycol, had been formulated 5 years previously. Relief from pain was reported with 14 days of use and continues to be applied when pain recurs.

### Example 9. Oral Administration of a 5 mg/ml Sterile Cobra Venom Liquid in Subject with Chronic Back Pain

From a stock solution of 400 mg/ml of sterile filtered cobra venom, a dilution was prepared by suspending 0.125 ml (50 mg) of the stock solution in 10 ml of saline, to reach a final venom concentration of 5 mg/ml.

A subject with chronic back pain was administered the 5 mg/mL product prepared as described above, suspended in 10 ml of saline, by mouth. The reported taste was very unpleasant, provoking lacrimation and coughing. The unpleasant aftertaste persisted for some time, accompanied by a slight feeling of nausea, which may have been due to drinking the saline, rather than being attributable to the venom. The subject noted that his throat was tender and had a scratchy feeling similar to that of a sore throat treated with a numbing agent. The subject reported that stiffness in the back was noticeable, but not back pain. The subject also noted eyelids feeling heavy. A slight headache was noted 90 minutes after ingestion of the solution that persisted for 8 hours. Throat symptoms were reported to be back to normal after 4 hours. No intestinal disturbances were reported.

### Example 10. Oral Administration of a 0.333 mg/ml) Sterile Cobra Venom Liquid to a Subject

From a stock solution of 350 mg/ml of sterile filtered cobra venom, an aliquot of 0.143 ml (50 mg) was suspended in 10 ml of purified water (final concentration 5 mg/ml) with 1 minute of secussion. This solution was mixed with 140 ml of pure orange juice giving a final concentration of 0.333 mg/ml. The formulation as described was administered to a subject with chronic back pain.

Upon ingestion, the subject reported no adverse effects save for a minor taste sensation in the mouth. None of the side effects traditionally associated with oral venom ingestion—such as esophageal irritation, lacrimation, coryza (acute inflammation of the mucous membrane of the nasal cavities; head cold), or intestinal disturbances were reported. Importantly, the same amount of venom drug was delivered (i.e., 50 mg) as in Example 1 above, however the volume of liquid was much greater (150 ml vs. 10 ml.) This result indicated that the same dosage (50 mg venom protein) that previously caused unacceptable side effects was completely tolerable when delivered in a much higher volume of liquid.

### Example 11. Oral Administration of a 1 mg/ml Sterile Cobra Venom Liquid in a Subject with Chronic Back Pain

The subject with chronic back pain of Example 9 was administered 0.0125 ml (5 mg) of a 400 mg/ml mother tincture of sterile filtered cobra venom solution, taken orally in 5 ml water (final concentration 1 mg/ml).

At the time of administration, the subject's back pain was estimated to be 4-5 on a scale of 1-10. The patient reported that the taste of the diluted solution was not nearly as harsh as before, even with the solution being rinsed around in the mouth before swallowing. The subject reported, however, that the taste worsened over time. Ninety minutes after administration, the patient reported a pain level of 0.5-1, with no adverse effects. Second and third administrations of a 5 mg dose at 8 and 24 hours after the first administration resulted in no adverse responses.

### Example 12. Oral Administration of a 0.4 mg/ml Sterile Cobra Venom Liquid in a Subject with Chronic Back Pain

A subject, while experiencing back pain at a level of 3-4 on a scale of 10, was administered an oral cobra venom product prepared 0.4 mg/ml in water.

The subject noted taste deterioration, and throat sensations as described above. A dull headache was noted 90 minutes later. Back pain was reduced to 1 to 1.5. Twelve hours later, the subject reported that the headache persisted, but the backache was reduced to a pain level of 0.5 on a scale of 1-10. Second and third doses of the formulation were taken on the second and third days, respectively. No adverse effects were noted, including absence of the characteristic throat irritation associated with previous homeopathic formulations of cobra toxin. Back pain levels were reported to be less than 0.5. No gastrointestinal upset was experienced.

### Example 13. Oral Administration of a 0.07 mg/ml Sterile Cobra Venom Beverage in a Subject with Chronic Back Pain

The subject in this Example was experiencing back pain at a level reaching 7-8 upon standing and settling in around 5. A beverage of cobra venom was prepared by adding 0.01 ml (3.5 mg) of a 350 mg/ml sterile cobra venom solution to purified water containing 5% pure lime juice, and 0.2% citric acid, made up to a final volume of 50 ml. Final concentration of the venom in the beverage solution was 0.07 mg/ml.

The subject reported minor irritation to the throat, although it was deemed to possibly have been attributable to either the venom or the lime juice. Within 1 hour, the subject's pain level was reduced to 3-4, and the subject could stand up and sit down easily. The subject was also able to touch his toes easily, which was usually not the case, suggesting some relaxation of muscles. Seven hours post administration, the pain level was further reduced to a level of 2-3. The subject also reported a significant improvement in sleep quality. Notably, the characteristic headache, typically experienced with the liquid formulations, failed to appear after ingestion of the beverage formulation. No gastrointestinal upset was experienced.

### Example 14. Oral Administration of 0.035 mg/ml (“Homeopathic 4×”) Sterile Cobra Venom by Oral Spray in Subjects with Chronic Back Pain

A formulation of sterile cobra venom at a concentration of 0.035 mg/mL was prepared and packaged in pump dispensers. The product was provided to six subjects with various types of chronic pain, with instructions to administer two sprays every 3-4 hours daily.

In general, a satisfactory reduction in pain was achieved in over 70% of the subjects. No gastrointestinal upset was reported, although a minor esophageal irritation was experienced, described as a dryness which diminished with continued use. In no case was the esophageal irritation sufficiently uncomfortable to discourage continued use of the pain relief product.

### Example 15. Oral Administration of a 0.07 mg/ml (“4×”) Sterile Cobra Venom by Oral Spray in Subjects with Chronic Back Pain

A formulation of cobra venom at 4× with citric acid, flavoring and methyl paraben was prepared in the manner described in Example 5 and packaged in pump dispensers. In this case, however, the final concentration of venom in the spray formulation was 0.07 mg/ml. If fractions were permitted, this formulation could be described as equivalent to a “3.8×” homeopathic formulation, as compared with the 4× formulation of Example 7.

This product was provided to 20 subjects with various types of chronic pain, who were instructed to take two sprays every 3-4 hours daily. In this group as well, a satisfactory reduction in pain was achieved in over 70% of the subjects. No gastrointestinal upset was reported, although minor esophageal irritation was experienced, as noted by subjects described in Example 6.

### Example 16. Oral Administration of 0.175 mg/ml Sterile Cobra Venom by Oral Spray in Subjects with Chronic Back Pain

A formulation of sterile cobra venom was prepared at a final concentration of venom in this formulation was 0.175 mg/ml. The spray formulation included citric acid, flavoring and methyl paraben, and was packaged in pump dispensers.

The product was provided to eight subjects with various types of chronic pain. Patients were instructed to take two sprays every 3-4 hours daily. In this instance, a satisfactory reduction in pain was achieved in over 90% of the subjects. Improved pain response was noted over the previous 4× formulation. No gastrointestinal upset was reported, although minor esophageal irritation as described above was observed. Nevertheless, this irritation was not severe enough to cause the subjects to discontinue use of the product.

### Example 17. Oral Administration of Cobra Venom at 0.14 mg/mL

A male subject, suffering with chronic back pain, was provided with a batch of cobra venom oral spray that had been formulated 5 years previously using the formula described in Example 3. The subject reported that pain relief was equivalent to a new batch of product, thereby confirming the stability of the formulation.

1. An analgesic composition comprising a sterile cobra venom solution admixed with a food-grade preservative and a polyol stabilizer in a metered pump configured to deliver the composition as an oral spray or jet.

2. The analgesic composition according to claim 1, wherein the sterile cobra venom solution has been sterilized by filtration through a filter having a pore size of about 0.45 μm or less.

3. The analgesic composition according to claim 1, wherein the sterile cobra venom has been sterilized by prolonged low-heat treatment or by pasteurization.

4. The analgesic composition according to claim 1, wherein the sterile cobra venom solution has a final homeopathic formulation of 3×.

5. The analgesic composition according to claim 4, wherein the protein concentration of the sterile cobra venom solution is from about 0.035 mg/ml to about 0.35 mg/ml.

6. The analgesic composition according to claim 1, wherein the sterile cobra venom solution has a final homeopathic formulation of 4×.

7. The analgesic composition according to claim 6, wherein the protein concentration of the sterile cobra venom solution is from about 0.0035 mg/ml to about 0.035 mg/ml.

8. The analgesic composition according to claim 1, wherein the sterile cobra venom solution has a final homeopathic formulation of 5×.

9. The analgesic composition according to claim 8, wherein the protein concentration of the sterile cobra venom solution is from about 0.00035 mg/ml to about 0.0035 mg/ml.

10. A method for the alleviating pain in a human and/or animal subject, the method comprising orally administering a composition comprising a sterile cobra venom solution admixed with a food-grade preservative and a polyol stabilizer, the venom solution having a homeopathic formulation of from about 3× to about 5× and the step of administering being performed using a metered pump configured to deliver the composition as an oral spray or jet.

11. The method according to claim 10, wherein the metered pump delivers a volume of solution in the range of 0.05 ml to 1 ml.

12. The method according to claim 11, wherein the metered pump delivers a volume of solution in the range of 0.1 ml to 0.2 ml.

13. The analgesic composition of claim 1, wherein the polyol comprises glycerol, propylene glycol, xylitol or combinations thereof.

14. The analgesic composition of claim 13, wherein the shelf life protein stability at room temperature refrigeration of is about 5 years.

15. The analgesic composition of claim 13, wherein the shelf life protein stability under refrigeration of is about 10 years.

16. The method of claim 10, wherein the polyol comprises glycerol, propylene glycol, xylitol or combinations thereof.

17. The method of claim 16, wherein the shelf life protein stability at room temperature refrigeration of is about 5 years.

18. The method of claim 16, wherein the shelf life protein stability under refrigeration of is about 10 years.
